(19) United States
(12) Patent Application Publication
(10) Pub. No.: US 2004/0023948 A1
(43) Pub. Date: Feb. 5, 2004

(54) Fast-dispersing dosage form containing 5-HT1 agonists

(76) Inventors: Richard David Green, Canterbury (GB); Jonathan Lacy, Genthod (CH); Nicholas Mallard, Bristol (GB); Edward Johnson, Berkshire (GB)

Correspondence Address: Andrew G. Rozycki, Cardinal Health, Inc., 7000 Cardinal Place, Dublin, OH, 43017, US

(21) Appl. No.: 10/375,560

(22) Filed: Feb. 26, 2003

Related U.S. Application Data
(63) Continuation-in-part of application No. 09/408,595, filed on Sep. 23, 1999, now abandoned.
Application No. 09/408,595 is a continuation of application No. PCT/GB98/00885, filed on Mar. 24, 1998.

(30) Foreign Application Priority Data
Mar. 24, 1997 (GB) 9706089.1

(51) Int. Cl. A61K 31/55; A61K 31/506

(52) U.S. Cl. 514/216; 514/252.15

(57) ABSTRACT

This invention relates to a pharmaceutical composition for oral administration comprising a carrier and, as an active ingredient, a 5-HT₁ agonist, characterized in that the composition is formulated to reduce pre-systemic metabolism of said 5-HT₁ agonist. A process for preparing such a composition and the use of such a composition for the treatment of anxiety, depression, attention deficit disorder and/or panic disorders and/or as a memory enhancer are also provided.

### RELATED APPLICATION DATA

This Application is a continuation-in-part of U.S. patent application Ser. No. 09/408,595; filed Sep. 23, 1999 which is a Continuation of International Application No. PCT/GB98/00885, filed Mar. 24, 1998 (claiming priority from British Application No. 9706089.1, filed Mar. 24, 1997), now pending, which are hereby incorporated by reference.

### FIELD OF THE INVENTION

This invention relates to a pharmaceutical composition, a process for preparing such a composition and the use of such a composition for the treatment of anxiety, depression, attention deficit disorder and/or panic disorders, sleep apnea and/or related respiratory disorders and/or substance addition, especially alcohol abuse, the treatment and/or prophylaxis of incontinence disorders, inducing immunosuppression and/or treating immune disorders, the alleviation of extrapyramidal motor disorders and/or as a memory enhancer.

### BACKGROUND OF THE INVENTION

Buspirone (8-[4-[4-(2-pyrimidinyl)-1-piperazinyl]butyl]-8-azaspiro[4.5]decane-7,9-dione) has been shown to be effective in the treatment of anxiety. The mechanism of action of buspirone has not been fully elucidated. However, it is known that buspirone is a 5-HT₁ agonist and, in particular, a potent 5-HT_{1A} agonist and it is thought that it is the action at these receptors which may account for its anxiolytic activity.

Buspirone is currently administered orally in the form of a conventional tablet which is scored in a manner which provide for it to be broken into halves or thirds along breaklines, thus allowing for some titration of the dose. However, each tablet or portion thereof is designed to be swallowed whole. Doses range from 15 to 60 mg per day and may be delivered as 2 or 3 divided doses. When administered in this way, buspirone is absorbed from the gastrointestinal tract, that is, the stomach, the small intestine and the proximal large intestine (colon), into the hepatic portal system and is presented to the liver before reaching the systemic circulation. The liver is known to be the principal site for conversion of active buspirone into metabolites and, indeed, buspirone is rapidly metabolized by the liver into a large number of metabolites. In a radio-label study in man, twelve metabolites of buspirone were isolated from urine (see H. K. Jajoo et al., Drug Metabolism and Disposition, (1989), 17, 634-640). However, only one of these metabolites, 1-pyrimidinylpiperazine (1-PP), has been reported to possess any potential therapeutic activity and this compound is said to possess, at most, only 20% of the activity of unchanged buspirone as determined by the Vogel conflict test in rats (see R. E. Gammans et al., Am. J. Med., (1986), 80 (suppl.3B), 41-51).

The mean systemic availability of unchanged buspirone is thought to be about 4% after conventional oral administration and the plasma levels of this drug are said to exhibit great variability. This latter effect has been attributed to differences between individuals in pre-systemic metabolism, that is, metabolism in the gastrointestinal tract, in the membranes lining the gastrointestinal tract and also in the liver. However, it is clear that the clinical effectiveness of buspirone is compromised by the extensive pre-systemic metabolism of this drug which occurs following conventional oral administration.

Buspirone is an example from a class of compounds known as the azapirones which have been shown to be effective in the treatment of anxiety. Other azapirones include 4,4-dimethyl-1-[4-[4-(2-pyrimidinyl)-1-piperazinyl]butyl]-2,6-piperidinedione (gepirone), 2-[4-[4-(2-pyrimidinyl)-1-piperazinyl]butyl]-1,2-benzisothiazol-3(2H)-one 1,1-dioxide (ipsapirone), (3aα,4α,4αβ, 6αβ, 7α,7aα)-3a,4,4a,6a,7,7a-hexahydro-2-[4-[4-(2-pyrimidinyl)-1-piperazinyl]butyl]-4,7-etheno-1H-cyclobut[f]isoindole-1,3(2H)-dione (zalospirone), 3-butyl-7-[4-[4-(2-methoxyphenyl)-1-piperazinyl]butyl]-9,9-dimethyl-3,7-diazabicyclo[3.3.1 ]nonane-2,4,6,8-tetrone(umespirone), (S)-8-[4-[(3,4-dihydro-5-methoxy-2H-1-benzopyran-3-yl)propylamino]butyl]-8-azaspiro[4.5]decane-7,9-dione (alnespirone), 6-(3-chlorophenoxy)-2-methyl-1-oxa-4-azaspiro[4.5]decan-3-one (enilospirone), octahydro-3-[4-[4-(2-pyrimidinyl)-1-piperazinyl]butyl]-1,5-methano-6,7,9-metheno-1H-pentaleno[1,2-d]azepine-2,4(3H,5H)-dione (WY-48723),4-[4-[4-(2-pyrimidinyl)-1-piperazinyl]butyl]-1,4-benzoxazepine-3,5 (2H,4H)-dione (SUN-8399), (3aα,4β,7β,7aα)-hexahydro-2-[4-[4-(2-pyrimidinyl)-1-piperazinyl]butyl]-4,7-methano-1H-isoindole-1,3(2H)-dione 2-hydroxy-1,2,3-propanetricarboxylate (1:1) (tandospirone) and salts thereof. All these compounds act as agonists at 5-HT₁ receptors, particularly 5-HT_{1A} receptors, and, like buspirone, are subject to extensive pre-systemic metabolism. Other compounds which interact with 5-HT₁ receptors (5-HT₁ agonists) include (+)—N-[2-[4-[2,3-dihydro-2-(hydroxymethyl)-1,4-benzodioxin-5-yl]-1-piperazinyl]ethyl]-4-fluoro-benzamide (flesinoxan), 2-[4-[4-(4-chloro-1H-pyrazol-1-yl)butyl]-1-piperazinyl]pyrimidine (lesopitron) (R)-3,4-dihydro-N-(1-methylethyl)-3-((1-methylethyl)propylamino)-2H-1-benzopyran-5-carboxamide (ebalzotan), N-[2-[4-(2-pyrimidinyl)-1-piperazinyl]]]adamantane-1-carboxamide (adatanserin), (R)-4-(dipropylamino)-1,3,4,5-tetrahydro-benz(cd)indole-6-carboxamide (LY-228729), F-8910-RS, (−)-cis-3-propyl-2,3,3a(R),4,5,9b-hexahydro-14-benz[e]indole-9-carboxamide (U-93385), 4-[2-[4-(naphthalen-1-yl)piperazin-1-yl)ethyl]quinolin-2(1H)one (SL-87.0765), 2-[4-[4,4-bis(4-fluorophenyl)butyl]-1-piperazinyl-3-pyridinecarboxylic acid methyl ester (FG-5893), 4-fluoro-N-[2-[4-[7-methoxy-1-naphthyl]piperazin-1-yl]ethyl]benzamide (S-14506), 5-methoxy-3-[4-(4-(4-methoxyphenyl)-1-piperazinyl)butyl]indole (EMD-56551),(−)-N-[2-(8-methyl-1,4-benzodioxan-2-ylmethylamino)ethyl]adamantane-1-carboxamide (HT-90B), F-92502-CN, 2-[4-[4-(4-nitropyrazol-1-yl)butyl]piperazin-1-yl]pyrimidine (E-4414), 5-[3-[[(2S)-1,4-benzodioxan-2-ylmethyl]-amino]propoxy]-1,3-benzodioxolane (MKC-242), 4-methyl-2-[4-(4-(pyrimidin-2-yl)-piperazino)butyl]-2H,4H-1,2,4-triazin-3,5-dione (F-12439), 1-(2-(4-(3-trifluoromethylphenyl)piperazin-1-yl)ethyl)benzimidazol-[1H]-2-one (BIMT-17), LY-39, SL-88.0338, 1,2,3,6-tetrahydro-1-[2-(2-naphthalenyl)ethyl]-4-[3-(trifluromethyl)phenyl]pyridine (SR-57746A), 1-(9H-fluoren-2-yl)-2-(1H-imidazol-1-yl)ethanone (LY-175644), (+/−)trans-2-(4-(3a,4,4a,6a,7,7a-hexahydro-4,7-etheno-1H-cyclobut[f]isoindol-1,3-dionyl)butyl-9-methoxy-2,3,3a,4,5,9b-hexahydro-1H-benz[e]isoindol (A-74283), 1,3,4,5-tetrahydo-6-methoxy-N,N-dipropylbenz[cd]indol-4-amine (Bay-r-1531), 4-(4-methyl-1-piperazinyl)-7-(trifluoromethyl)-pyrrolo[1,2-a]quinoxaline (Z)-2-butendioate (1:2) (CGS-12066B), trans-1,3,4,4a,5,10b-hexahydro-10-methoxy-4-propyl-2H-[1]benzopyrano[3,4-b]pyridine (CGP-50281), N-propyl-N-[2-(4-fluorobenzamido)ethyl]amino-5,6,7,8-tetrahydroquinoline (WAY-100012), 3a,4,4a,6a,7,7a-hexahydro-2-[4-[4-(2-pyrimidinyl)-1-piperazinyl]butyl]-4,7-ethenocyclobuta[f]-1,2-benzisothiazol-3(2H)-one 1,1-dioxide, cis-7-chloro-10-methoxy-5a,10b-dihydro-3N-n-propyl-6H-indeno[1,2-d]azepine, 1-cyclohexyl-3-[4-[4-(2,3-dihydro-2,2-dimethylbenzofuran-7-yl)-1-piperazinyl]-1-butyl]-2-imidazolidinone, 1-[3-amino-2H-1-benzopyran-2-one-8-yl)piperazine, 4-(1-methylethyl)-2-[3-(trifluoromethyl)-phenyl]morpholine (oxaflozane), N,5-dimethyl-10-dibenz-(b,f)azepine-ethanamine (RU-5031), 1,4-dihydro-3-(1,2,3,6-tetrahydro-4-pyridinyl)-5H-pyrrolo[3,2-b]-pyridin-5-one (CP-93129), N-methyl-3-(1-methyl-4-piperidinyl)-1H-indole-5-ethanesulphonamide (naratriptan), 3-[3-[4-(5-methoxy-4-pyrimidinyl)-1-piperazinyl]propyl]-N-methyl-1H-indole-5-methanesulphonamide (avitriptan), N,N-dimethyl-5-(1H-1,2,4-triazol-1-ylmethyl)-1H-indole-3-ethanamine (rizatriptan), N-4-[[5-[3-(2-aminoethyl)-1H-indol-5-yl]-1,2,4-oxadiozol-3-yl]methyl]phenyl]methanesulphonamide (L-694247), IS-159, 1-(((3-(2-(dimethylamino)ethyl)-1H-indol-5-yl)methyl)sulphonyl) pyrrolidine (almotriptan), 4-((3-(2-(dimethylamino)ethyl)-1H-indol-5-yl)methyl-(S)-2-oxazolidinone (zolmitriptan), 3-[2-(dimethylamino)-ethyl]-N-methyl-1H-indole-5-methanesulphonamide (sumatriptan),3-((1-methyl-2-pyrrolidinyl)methyl)-5-(2-(phenylsulphonyl)ethyl)-(R)-1H-indole (eletriptan), (R)-(+)-3-(methylamino)-1,2,3,4-tetrahydro-9H-carbazole-6-carboxamide (VML-251), L-0076, ALX-0625, (R)-N-methyl-[3-(1-methyl-2-pyrrolidinyl)-1 H-indol-5-yl)methanesulphonamide (CP-122288), 3-[3-[4-(5,6-dimethoxypyrimidin-4-yl)piperazin-1-yl]propyl]-N-methyl-1H-indol-5-yl-methylsulphonamide (VS-395), L-747201, LY-334370 and salts thereof.

It would be highly desirable from a clinical point of view to find a way of administering such 5-HT₁ agonists so that the bioavailability of the active ingredient is enhanced and the variability in plasma levels caused by differences in pre-systemic metabolism is reduced.

According to the present invention, there is therefore provided a pharmaceutical composition for oral administration comprising a carrier and, as an active ingredient, a 5-HT₁ agonist, characterized in that the composition is formulated to reduce pre-systemic metabolism of the 5-HT₁ agonist.

If pre-systemic metabolism of the active ingredient is to be reduced, it is important that the active ingredient is absorbed into the systemic circulation at a site which enables the active ingredient to avoid entering the portal circulation to the liver and thus avoid extensive metabolism by the liver (the so-called “first pass effect”). Since absorption from the gastrointestinal tract is known to result in the active ingredient entering the hepatic portal system to the liver, one option for reduction of pre-systemic metabolism is to promote absorption from sites before the active ingredient reaches the gastrointestinal tract. Accordingly, it is preferred that the composition of the invention is formulated to promote pre-gastric absorption of the 5-HT₁ agonist.

### DETAILED DESCRIPTION OF THE INVENTION

The term “pre-gastric absorption” is used to refer to absorption of the active ingredient from that part of the alimentary canal prior to the stomach and includes buccal, sublingual, oropharyngeal and esophageal absorption.

It is envisaged that such pre-gastric absorption will occur primarily across the mucous membranes in the mouth, pharynx and esophagus. Accordingly, it is preferred that the composition of the invention is formulated to promote absorption of the active ingredient through the buccal, sublingual, pharyngeal and/or esophageal mucous membranes.

It is therefore preferred that the composition of the invention should be in the form which sustains the active ingredient in contact with the buccal, sublingual, pharyngeal and/or esophageal mucous membranes.

Preferably, the composition of the invention is in the form of a viscous emulsion, syrup or elixir, a softgel, lozenge, aqueous or non-aqueous drops or other dosage form designed to release the active ingredient in a controlled manner to saliva or to the buccal, pharyngeal and/or esophageal mucous membranes, a fast-dispersing dosage form designed rapidly to release the active ingredient in the oral cavity, or a bioadherent system.

The term “bioadherent system” refers to a solid or liquid dosage form which, at body temperature, exhibits controlled release and bioadherence characteristics. This type of dosage form may be an emulsion which is water in oil in nature and whose internal phase is greater than that of the external phase. Examples of such bioadherent systems may be found in U.S. Pat. No. 5,055,303.

Of the dosage forms listed above, fast-dispersing dosage forms are particularly preferred since they will disintegrate rapidly in the mouth without the need for water, or other liquid, to aid swallowing. Such fast-dispersing dosage forms are therefore more convenient and easier for patients to take than conventional oral dosage forms. Also, since no water, or other liquid, is required to take such fast-dispersing dosage forms, the active ingredient is presented for absorption at a higher concentration than with conventional oral dosage forms. Thus, use of such fast-dispersing dosage forms may allow a reduction in the dose required to achieve the desired therapeutic effect which, in turn may result in a reduction in the incidence and/or severity of side effects. More reproducible plasma levels of the active ingredient may also be achieved with such dosage forms.

Since such fast-dispersing dosage forms produce a concentrated solution of the active ingredients in the saliva of the mouth, it is also possible that, when swallowed, this concentrated solution coats the stomach mucosa more effectively than a conventional drug dissolved in water and this may increase the rate of absorption of the active ingredient. Moreover, absorption from the highest part of the stomach may also by-pass the hepatic portal vein and hence produce a higher level of the active ingredient in the plasma.

One example of a fast-dispersing dosage form is described in U.S. Pat. No. 4,855,326 in which a melt spinnable carrier agent, such as sugar, is combined with an active ingredient and the resulting mixture spun into a “candy-floss” preparation. The spun “candy-floss” product is then compressed into a rapidly dispersing, highly porous solid dosage form.

U.S. Pat. No. 5,120,549 discloses a fast-dispersing matrix system which is prepared by first solidifying a matrix-forming system dispersed in a first solvent and subsequently contacting the solidified matrix with a second solvent that is substantially miscible with the first solvent at a temperature lower than the solidification point of the first solvent, the matrix-forming elements and active ingredient being substantially insoluble in a second solvent, whereby the first solvent is substantially removed resulting in a fast-dispersing matrix.

U.S. Pat. No. 5,079,018 discloses a fast-dispersing dosage form which comprises a porous skeletal structure of a water soluble, hydratable gel or foam forming material that has been hydrated with water, rigidified in the hydrated state with a rigidifying agent and dehydrated with a liquid organic solvent at a temperature of about 0C or below to leave spaces in place of hydration liquid.

Published International Application No. WO 93/12769 (PCT/JP93/01631) describes fast-dispersing dosage forms of very low density formed by gelling, with agar, aqueous systems containing the matrix-forming elements and active ingredient, and then removing water by forced air or vacuum drying.

U.S. Pat. No. 5,298,261 discloses fast-dispersing dosage forms which comprise a partially collapsed matrix network that has been vacuum dried above the collapse temperature of the matrix. However, the matrix is preferably at least partially dried below the equilibrium freezing point of the matrix.

Published International Application No. WO 91/04757 (PCT/US90/05206) discloses fast-dispersing dosage forms which contain an effervescent disintegration agent designed to effervesce on contact with saliva to provide rapid disintegration of the dosage form and dispersion of the active ingredient in the oral cavity.

U.S. Pat. No. 5,595,761 discloses a particulate support matrix for use in making a rapidly dissolving tablet, comprising:

a first polypeptide component having a net charge when in solution, e.g., non-hydrolyzed gelatin;

a second polypeptide component having a net charge of the same sign as the net charge of the first polypeptide component when in solution e.g. hydrolyzed gelatin; and

a bulking agent, and wherein the first polypeptide component and the second polypeptide component together comprise about 2% to 20% by weight of the particulate support matrix and wherein the bulking agent comprises about 60% to 96% by weight of the particulate support matrix; and

wherein the second polypeptide component has a solubility in aqueous solution greater than that of the first polypeptide component and wherein the mass:mass ratio of the first polypeptide component to the second polypeptide component is from about 2:1 to about 1:14; and

wherein when the support matrix is introduced into an aqueous environment the support matrix is disintegrable within less than about 20 seconds.

U.S. Pat. No. 5,576,014 discloses fast-dispersing dosage forms which dissolve intrabuccally and which comprise compressed moldings formed from granules comprising a saccharide having low moldability which has been granulated with a saccharide having high moldability. The resulting compressed moldings show quick disintegration in the buccal cavity.

EP-B-0690747 describes particles comprising an excipient forming a matrix and at least one active ingredient uniformly distributed in the mass of the matrix which are prepared by a process comprising the steps of preparing an homogenous pasty mixture with a viscosity below 1 Pa.s, measured at room temperature (15-20° C.), from at least one active ingredient, a physiologically acceptable hydrophilic excipient and water; extruding the resulting homogenous mixture and cutting the extrudate to give moist particles; freezing the resulting particles as they fall under gravity through a stream of inert gas at a temperature below 0C; and drying the particles by freeze drying.

Australian Patent No. 666,666 discloses a rapidly disintegratable multiparticulate tablet having a mixture of excipients in which the active substance is present in the form of coated microcrystals or optionally coated microgranules. Such tables disintegrate in the mouth in an extremely short time, typically less than 60 seconds.

U.S. Pat. No. 5,382,437 discloses a porous carrier material having sufficient rigidity for carrying and administering an active material which is capable of rapid dissolution by saliva and which is formed by freezing a liquefied ammonia solution comprising liquid ammonia, a liquid ammonia-soluble gel or foam material and a rigidifying agent for the gel or foam material and a rigidifying agent for the gel or foam material selected from the group consisting of a monosaccharide, a polysaccharide and combinations thereof, and deammoniating the frozen material thus formed by causing material transfer of ammonia from the frozen state to the gas state thereby leaving spaces in the carrier material in place of the frozen ammonia.

Published International Application No. WO 93/13758 (PCT/US92/07497) describes tablets of increased physical strength which are prepared by combining and compressing a meltable binder, excipients and a pharmaceutically active agent into a tablet, melting the binder in the tablet and then solidifying the binder. In one embodiment, a disintegrating agent is utilized to increase the disintegration rate of the tablet after oral intake. In another embodiment, a volatilizable component is used to form porous tablets. Some embodiments disintegrate in the mouth in less than 10 seconds.

U.S. Pat. Nos. 3,885,026 and 4,134,943 also disclose fast-dispersing porous tablets and a method for increasing their physical strength by first compressing the tablet and then volatilizing a readily volatilizable solid adjuvant incorporated in the tablet to attain the desired porosity.

EP-A-0601965 describes a shearform matrix material which can be used, inter alia, to deliver a pharmaceutical active agent. The shearform matrix is formed by increasing the temperature of a feedstock which includes a solid non-solubilized carrier material to the point where it will undergo internal flow with the application of a fluid shear force, ejecting a stream of the heated feedstock thus formed under pressure from an orifice and then subjecting the feedstock to disruptive fluid shear force which separates the flow of feedstock into multiple parts and transforms the morphology of the feedstock.

U.S. Pat. No. 5,683,720 discloses discrete particles containing a pharmaceutically active agent which can be fast-dispersing and are formed by subjecting a solid, organic feedstock to liquiflash conditions whereby the feedstock is transformed instantaneously from solid to liquiform to solid, liquiform being a transient condition in which the feedstock has substantially unimpeded internal flow. Shear force is then imparted to the liquiform feedstock in an amount sufficient to separate tiny masses of feedstock which then solidify as discrete particles.

The term “fast-dispersing dosage form” therefore encompasses, but is not limited to, all the types of dosage form described in the preceding paragraphs. However, it is particularly preferred that the fast-dispersing dosage form is of the type described in U.K. Patent No. 1548022, that is, a solid fast-dispersing dosage form comprising a network of the active ingredient and a water-soluble or water-dispersible carrier which is inert towards the active ingredient, the network having been obtained by subliming solvent from a composition in the solid state, that composition comprising the active ingredient and a solution of the carrier in a solvent.

It is preferred that the composition of the invention disintegrates within 1 to 60 seconds, more preferably 1 to 30 seconds, especially 1 to 10 seconds, and particularly 2 to 8 seconds, of being placed in the oral cavity.

In the case of the preferred type of fast-dispersing dosage form described above, the composition will preferably contain, in addition to the active ingredient, matrix forming agents and secondary components. Matrix forming agents suitable for use in the present invention include materials derived from animal or vegetable proteins, such as the gelatins, dextrins and soy, wheat and psyllium seed proteins; gums such as acacia, guar, agar and xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

Other matrix forming agent suitable for use in the present invention include sugars such as mannitol, dextrose, lactose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine.

One or more matrix forming agents may be incorporated into the solution or suspension prior to solidification. The matrix forming agent may be present in addition to a surfactant or to the exclusion of a surfactant. In addition to forming the matrix, the matrix forming agent may aid in maintaining the dispersion of any active ingredient within the solution or suspension. This is especially helpful in the case of active agents that are not sufficiently soluble in water and must, therefore, be suspended rather than dissolved.

Secondary components such as preservatives, antioxidants, surfactants, viscosity enhancers, coloring agents, flavoring agents, pH modifiers, sweeteners or taste-masking agents may also be incorporated into the composition. Suitable coloring agents include red, black and yellow iron oxides and FD & C dyes such as FD & C blue No. 2 and FD & C red No. 40 available from Ellis & Everard. Suitable flavoring agents include mint, raspberry, licorice, orange, lemon, grapefruit, caramel, vanilla, cherry and grape flavors and combinations of these. Suitable pH modifiers include citric acid, tartaric acid, phosphoric acid, hydrochloric acid and maleic acid. Suitable sweeteners include aspartame, acesulfame K and thaumatin. Suitable taste-masking agents include sodium bicarbonate, ion-exchange resins, cyclodextrin inclusion compounds, adsorbates or microencapsulated actives.

Preferred compositions in accordance with this invention include a 5-HT₁ agonist, especially an azapirone or a salt thereof, as the active 5-HT₁ agonist. It is particularly preferred that the active 5-HT₁ agonist is buspirone or a salt thereof.

Buspirone which is absorbed by pre-gastric absorption or at a high rate across the stomach mucosa from a composition in accordance with this invention passes straight into the systemic circulatory system thereby avoiding first pass metabolism in the liver. Accordingly, the initial rapid production of unwanted, inactive metabolites is reduced and the bioavailability of active buspirone is increased. This results in a number of advantages. For instance, the increased bioavailability of active buspirone means that the dose of buspirone may be reduced while still producing the desired beneficial effect. This will result in a further decrease in the production of unwanted metabolites and reduction in the incidence and/or severity of side effects.

In the case of buspirone, the active ingredient preferably is present in the composition in an amount of from 0.5 to 30%, more preferably 1 to 20%, by weight of the composition. It is also preferred that the active ingredient is present in the composition in an amount of from 0.25 to 50 mg, more preferably 0.5 to 10 mg and, especially, 1 to 10 mg.

In the case of other 5-HT₁ agonists, these also will be present in concentrations which are clinically effective.

According to another aspect of the invention, there is provided a process for preparing a pharmaceutical composition as defined above which comprises bringing a carrier into association with the active ingredient.

In a further aspect, the invention provides the use of a fast-dispersing dosage form designed to release active ingredient rapidly in the oral cavity to deliver a 5-HT₁ agonist. A method of administering a 5-HT₁ agonist to a patient which comprises introducing into the oral cavity of the patient a composition as previously defined is also provided.

The invention also provides, in another aspect, a composition as defined above for use in the treatment of anxiety. The composition of the invention is also useful in the palliative treatment of anxiety neurosis, that is, neurosis with a preponderance of anxiety symptoms.

5-HT₁ agonist, especially the azapirones, have also been evaluated in the treatment of depression, attention deficit disorder and panic disorders and as memory enhancers. Also, the azapirones, especially buspirone, have been found to be useful in the treatment and/or prophylaxis of incontinence disorders associated with the gastrointestinal or urogenital tracts, such as urinary incontinence, fecal incontinence and urinary retention. Such compounds are also useful for inducing immunosuppression and/or treating immune disorders and are therefore useful in the treatment of conditions such as contact, atopic or eczematous dermatitis and Sjogren's syndrome (including secondary keratonconjunctivitis sicca), autoimmune diseases and diseases of known or unknown etiology having an immunological component or allergies, especially rheumatoid arthritis and, in particular, juvenile rheumatoid arthritis. Such compounds may also be used in the treatment of sleep apnea and related respiratory disorders, such as sudden infant death syndrome, and can also alleviate the symptoms of sleep apnea such as anxiety, depression, fatigue, malaise, irritability, anger and hostility.

In addition, azapirones such as buspirone can be used in the treatment of substance addiction. In this respect, substance addiction includes over-eating, eating disorders and the habitual use of alcohol, tobacco, marijuana, cocaine, opiates, methadone, amphetamine, methphenidate and related designer drugs. Such compounds are particularly useful in the treatment of alcohol abuse as they also reduce the craving for alcohol and can therefore be used for treating patients undergoing short term treatment of alcohol withdrawal and in chronic alcohol abusers. Use of such compounds in the treatment of alcohol abuse avoids the enhancement or continuation of sensory impairment, the risk of developing drug dependence and the unpleasant side effects of so-called aversion therapy. Moreover, such compounds produce behavioral modifications which include lessening of alcohol cravings and ingestion and improvement of social functioning. In addition, psychogenic symptoms, such as illness, anxiety, depression, clouded sensorium, hostility, violence and decreased cognition are alleviated.

Azapirones, particularly buspirone, are also useful for the alleviation of extrapyramidal motor disorders and can therefore be used to treat such conditions as Parkinson's disease, neuroleptic-induced extrapyramidal symptoms and tardive dyskinesia.

In addition to the above conditions, 5-HT₁ agonist have also been used in the treatment of social phobia, obsessive-compulsive disorder, migraine, cerebellar ataxia, levodopa-induced dyskinesias, Huntington's disease, central and peripheral neurodegenerative disorders, emesis, hypertension, hayfever, asthma and pruritis and to assist smokers in giving up smoking.

Of the 5-HT₁ agonists other than the azapirones, the 5-HT_{1D} agonists have been found to be especially useful in the treatment of migraine and related conditions.

According to a further aspect of the invention there is therefore provided the use of a composition as defined above for the manufacture of a medicament for the treatment of depression, attention deficit disorder, panic disorders, sleep apnea and/or related respiratory disorders and/or substance addiction, especially alcohol abuse, the treatment and/or prophylaxis of incontinence disorders, inducing immunosuppression and/or treating immune disorders, the alleviation of extrapyramidal motor disorders and/or as a memory enhancer.

The invention is further illustrated by the following examples.

### EXAMPLES

### Example 1

Preparation of a Fast-Dispersing Dosage Form of Buspirone Hydrochloride

(a) Preparation of Buspirone Hydrochloride 2.0% Dispersion

Gelatin (720 g) and mannitol (540 g) were dispersed in a portion of purified water (16 kg) by mixing thoroughly in the bowl of a vacuum mixer. The mix was then heated to 40° C.±2° C. and homogenized for ten minutes. The mix was cooled down to room temperature (20−24° C.). When cooled the glycine (180 g) and buspirone hydrochloride (360 g) was added. The mix was homogenized to ensure dissolution of glycine and the drug. Citric acid (54 g) was added gradually with stirring, to adjust the solution pH to 4.0. The remaining water (146 g) was added to the mixer and the bulk mix homogenized to ensure dissolution was complete.

(b) Preparation of Buspirone Hydrochloride 10 mg Units

500 mg of the buspirone hydrochloride 2.0% dispersion formed in (a) above was dosed into each one of a series of pre-formed blister pockets having a pocket diameter of 16 mm. The blister laminate comprised 200 μm PVC coated with 40 gsm PVdC. The product was frozen immediately in a liquid nitrogen freeze tunnel. The frozen product was then stored below −18° C. for a minimum of 40 hours prior to freeze-drying in a freeze drier using a drying temperature of +20° C. and a chamber pressure of 0.5 mbar. The freeze dried units were then inspected for the presence of critical defects and the remainder of the batch sealed with lidding foil consisting of the batch sealed with lidding foil consisting of a paper/foil laminate (20 μm aluminum).

Each blister was then coded with a batch number and overwrapped in a preformed sachet by placing the blister in the sachet and sealing the open end of the sachet completely. Each sachet was then labeled with the product name, batch number, date of manufacture and supplier's name.

Each dosage unit had the following composition:

| | | % by weight of |
|---|---|---|
| Ingredient | Weight (mg) | composition |
| Purified Water USP/EP* | 448.500 | 89.7 |
| Buspirone HC1 USP | 10.000 | 2.0 |
| Gelatin EP/USNF | 20.000 | 4.0 |
| Mannitol EP/USP | 15.000 | 3.0 |
| Glycine USP | 5.000 | 1.0 |
| Citric Acid | 1.500 | 0.3 |
| Total | 500.000 | 100.000 |

*Signifies removed during the lyophilization process.

### Example 2

Comparative Pharmacokinetic Study

The aim of this experiment was to compare the bioavailability of the buspirone hydrochloride formulation of Example 1 with the commercially available tablet formulation of buspirone hydrochloride sold under the registered Trade Mark “Buspar” by Bristol-Myers Pharmaceuticals, Bristol-Myers Squibb House, 141-149 Staines Road, Hounslow, Middlesex TW3 3JA, England.

An open label, randomized, crossover, volunteer study was performed as follows. Six fasted, healthy male subjects of ages between 18 and 40 years, giving written informed consent, underwent a thorough medical examination to establish their fitness to participate in the study. Subjects received study treatment in the order dictated by a predetermined randomization schedule. Subjects were given either the formulation of Example 1 or the “Buspar” formulation. Blood samples for determination of pharmacokinetic parameters were taken at baseline (immediately before drug administration), then after 0.25, 0.5, 0.75, 1, 1.5, 2, 2.5, 3, 4, 5, 6, 7, 8, 12 and 24 hours. The study procedures were repeated two weeks later, when subjects were crossed-over to receive their second drug administration. Buspirone hydrochloride was administered as single 20 mg doses (made up from 2×10 mg tablets) of the formulation of Example 1 or of the “Buspar” formulation.

HPLC-MS assays were performed to determine the concentration of buspirone and 1-pyrimidinylpiperazine (1-PP) in each of the blood plasma samples. The following pharmacokinetic parameters were determined for both analyzed substances: bioavailability (as measured as the area under the curve (AUC) of the drug concentration/time plot) and Cmax (the maximum plasma concentration achieved).

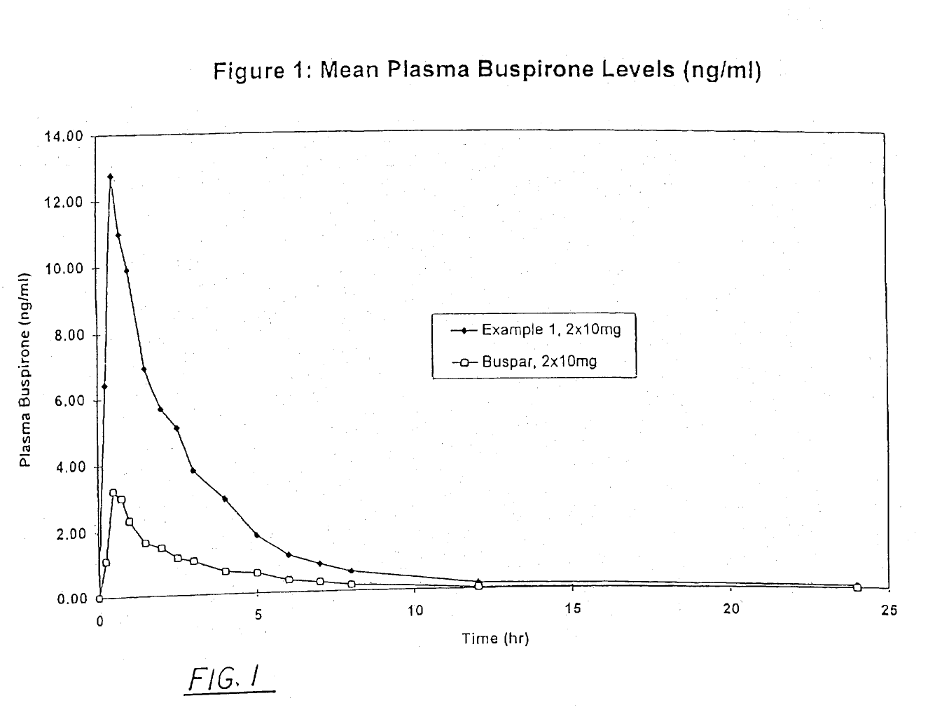

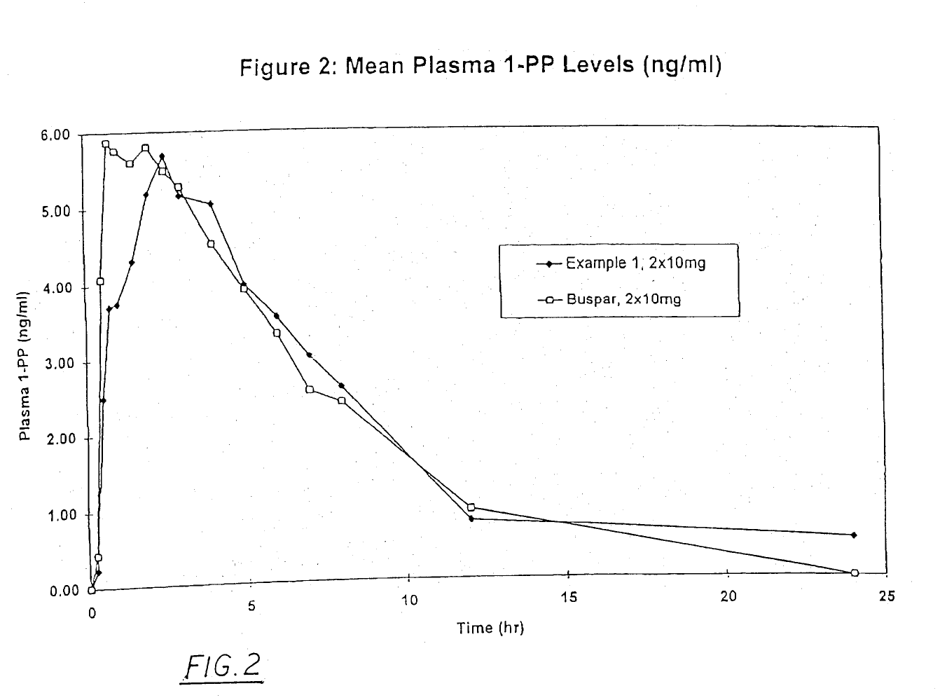

The results are shown in graphical form in FIGS. 1 and 2 where each figure is a plot of the concentration of a specific compound in a blood plasma sample versus the time at which the sample was taken for the formulation of Example 1 (Example 1) and the tablet formulation sold under the registered Trade Mark “Buspar” (Buspar). In FIG. 1, the specific compound is buspirone. In FIG. 2, the specific compound is 1-pyrimidinylpiperazone (1-PP).

The mean results are shown in numerical form in Table 1 below.

TABLE 1

| | | Buspirone | | 1-PP |
|---|---|---|---|---|
| | Cₘₐₓ | AUC_{(0-24 hr)} | Cₘₐₓ | AUC_{(0-24 hr)} |
| | (ng/ml) | (ng/ml.hr) | (ng/ml) | (ng/ml.hr) |
| Example 1, 20 mg | 13.0 | 33.4 | 6.5 | 46.5 |
| Buspar 20 mg | 3.6 | 8.0 | 6.8 | 37.9 |

From FIGS. 1 and 2 and Table 1, it is apparent that the bioavailability of buspirone from the formulation of Example 1 is about four times that of buspirone from the “Buspar” formulation despite the fact that both formulations contained the same amount of active ingredient. Also, the bioavailability of 1-pyrimidinylpiperazine (1-PP) is very similar for both formulations. However, in view of the much greater bioavailability of buspirone from the formulation of Example 1, it is envisaged that the dose of buspirone could significantly reduced thereby significantly reducing the quantity of unwanted metabolites and the incidence or severity of side effects while still achieving the desired levels of buspirone in plasma and hence the desired levels of buspirone in plasma and hence the desired therapeutic effect associated with this compound.

In Table 1, the ratio of the area under the plasma concentration-time curve (AUC) for buspirone and the AUC for 1-PP was 0.211 for the “Buspar” formulation, indicating clearly the extensive metabolism of buspirone when administered in a conventional tablet form. The corresponding AUC ratio for Example 1 in Table 1 was 0.718. This demonstrates that administration in the formulation of Example 1 results in a greater proportion of the administered dose of buspirone being absorbed in the unmetabolized form, and, indeed, it is apparent from FIG. 2 that metabolism of buspirone occurs more slowly for the formulation of Example 1 since the maximum amount of 1-PP for the formulation of Example 1 is observed about 2 hours later than that for the conventional “Buspar” formulation.

Pharmaceutical compositions of this invention will increase the ratio of the unchanged drug to the main metabolite's area under the plasma concentration-time curve (AUC) by at least 1.5 times and, most preferably, by at least 2 times.

### Examples 3 to 11

The following additional fast-dispersing dosage forms may be prepared according to the method of Example 1:-

### Example 3

| | | % by wt of |
|---|---|---|
| Ingredient | Weight (mg) | composition |
| Purified Water EP/USP* | 223.875 | 89.550 |
| Buspirone HC1 | 3.000 | 1.200 |
| Gelatin EP/USNF | 10.000 | 4.000 |
| Mannitol EP/USP | 7.500 | 3.000 |
| Glycine EP/USP | 2.500 | 1.000 |
| Banana Flavor | 0.625 | 0.250 |
| Raspberry Flavor | 0.625 | 0.250 |
| Aspartame EP/USNF | 1.8.75 | 0.750 |
| Total | 250.000 | 100.000 |

*Signifies removed during the lyophilization process.

### Example 4

| | | % by wt of |
|---|---|---|
| Ingredient | Weight (mg) | composition |
| Purified Water EP/USP* | 450.000 | 90.150 |
| Buspirone HCl | 3.000 | 0.600 |
| Gelatin EP/USNF | 20.000 | 4.000 |
| Mannitol EP/USP | 15.000 | 3.000 |
| Glycine EP/USP | 5.000 | 1.000 |
| Mint Flavour | 2.500 | 0.50 |
| Aspartame EP/USNF | 3.7505 | 0.750 |
| Total | 500.000 | 100.000 |

*Signifies removed during the lyophilization process.

### Example 5

| | | % by wt of |
|---|---|---|
| Ingredient | Weight (mg) | composition |
| Purified Water EP/USP* | 224.750 | 89.900 |
| Flesinoxan HCl | 4.000 | 1.600 |
| Gelatin EP/USNF | 11.250 | 4.500 |
| Mannitol EP/USP | 7.500 | 3.000 |
| Mint Flavor | 1.250 | 0.500 |
| Aspartame EP/USNF | 1.250 | 0.500 |
| Total | 250.000 | 100.000 |

*Signifies removed during the lyophilization process.

### Example 6

| | | % by wt of |
|---|---|---|
| Ingredient | Weight (mg) | composition |
| Purified Water EP/USP* | 666.875 | 88.917 |
| Gepirone HCl | 10.000 | 1.333 |
| Gelatin EP/USNF | 33.750 | 4.500 |
| Mannitol EP/USP | 26.250 | 3.500 |
| Grape Flavor | 3.750 | 0.500 |
| Raspberry Flavor | 3.750 | 0.500 |
| Aspartame EP/USNF | 5.625 | 0.750 |
| Total | 750.000 | 100.000 |

*Signifies removed during the lyophilization process.

### Example 7

| | | % by wt of |
|---|---|---|
| Ingredient | Weight (mg) | composition |
| Purified Water EP/USP* | 448.750 | 89.750 |
| Ipsapirone HCl | 5.000 | 1.000 |
| Gelatin EP/USNF | 20.000 | 4.000 |
| Mannitol EP/USP | 15.000 | 3.000 |
| Glycine USP | 2.500 | 0.500 |
| Citric Acid EP/USP | 2.500 | 0.500 |
| Licorice Flavor | 3.750 | 0.750 |
| Aspartame EP/USNF | 2.500 | 0.500 |
| Total | 500.000 | 100.000 |

*Signifies removed during the lyophilization process.

### Example 8

| | | % by wt of |
|---|---|---|
| Ingredient | Weight (mg) | composition |
| Purified Water EP/USP* | 225.625 | 90.250 |
| Alnespirone | 2.500 | 1.000 |
| Gelatin EP/USNF | 11.250 | 4.500 |
| Mannitol EP/USP | 7.500 | 3.000 |
| Grapefruit Flavor | 1.250 | 0.500 |
| Aspartame EP/USNF | 1.875 | 0.750 |
| Total | 250.000 | 100.000 |

*Signifies removed during the lyophilization process.

### Example 9

| | | % by wt of |
|---|---|---|
| Ingredient | Weight (mg) | composition |
| Purified Water EP/USP* | 649.375 | 86.583 |
| Sumatriptan Succinate | 35.000 | 4.667 |
| Gelatin EP/USNF | 31.875 | 4.250 |
| Mannitol EP/USP | 22.500 | 3.000 |
| Cherry Flavor | 3.750 | 0.500 |
| Aspartame EP/USNF | 7.500 | 1.000 |
| Total | 750.000 | 100.000 |

*Signifies removed during the lyophilization process.

### Example 10

| | | % by wt of |
|---|---|---|
| Ingredient | Weight (mg) | composition |
| Purified Water EP/USP* | 225.000 | 90.000 |
| Zolmitriptan | 5.000 | 2.000 |
| Gelatin EP/USNF | 10.000 | 4.000 |
| Mannitol EP/USP | 7.500 | 3.000 |
| Cherry Flavor | 1.250 | 0.500 |
| Aspartame EP/USNF | 1.250 | 0.500 |
| Total | 250.000 | 100.000 |

*Signifies removed during the lyophilization process.

### Example 11

| | | % by wt of |
|---|---|---|
| Ingredient | Weight (mg) | composition |
| Purified Water EP/USP* | 428.750 | 85.750 |
| Oxaflozane | 30.000 | 6.000 |
| Gelatin EP/USNF | 20.000 | 4.000 |
| Mannitol EP/USP | 15.000 | 3.000 |
| Lemon Flavor | 2.500 | 0.500 |
| Aspartame EP/USNF | 3.750 | 0.750 |
| Total | 500.000 | 100.000 |

*Signifies removed during the lyophilization process.

The invention has been described herein with reference to various specific and preferred embodiments and techniques. It will be understood, however, that reasonable variations and modifications of such embodiments and techniques are possible without significantly departing from either the spirit or scope of the invention. The text of patents, patent applications and publications referred to in this application are incorporated herein by reference.

What is claimed is:

1. A pharmaceutical composition for oral administration comprising a carrier and, as an active ingredient, a 5-HT₁ agonist, characterized in that the composition is a solid fast-dispersing dosage form which disintegrates within 1 to 10 seconds of being placed in the oral cavity, said solid fast-dispersing dosage form comprising a network of the active ingredient and a water-soluble or water-dispersible carrier which is inert toward the active ingredient, the network having been obtained by subliming solvent from a composition in the solid state.

2. A composition according to claim 1 in which the composition is formulated to promote pre-gastric absorption of said 5-HT₁ agonist.

3. A composition according to claim 1 in which the composition is formulated to promote absorption of said 5-HT₁ agonist through the buccal, sublingual, pharyngeal and/or esophageal mucous membrane.

4. A composition according to claim 1 in which the composition is formulated to promote absorption of said 5-HT₁ agonist through the stomach mucous membrane.

5. A composition according to claim 1 in which the 5-HT₁ agonist is selected from buspirone, gepirone, ipsapirone, zalospirone, umespirone, alnespirone, enilospirone, WY-48723, SUN-8399, tandospirone, flesinoxan, lesopitron, ebalzotan, adatanserin, LY-228729, F-8910-RS, U-93385, SL-87.0765, FG-5893, S-14506, EMD-56551, HT-90B, F-92502-CN, E-4414, MKC-242, F-12439, BIMT-17, LY-39, SL-88.0338, SR-57746A, LY-175644, A-74283, Bay-r-1531, CGS-12066B, CGP-50281, WAY-100012, 3a,4,4a,6a,7,7a-hexahydro-2-[4-[4-(2-pyrimidinyl)-1-[piperazinyl]butyl]-4,7-ethenocyclobuta[f]-1,2-benzisothiazol-3(2H)-one 1,1-dioxide, cis-7-chloro-10-methoxy-5a 10b-dihydro-3N-n-propyl-6H-indeno[ 1,2-d]azepine, 1-cyclohexyl-3-[4-[4-(2,3-dihydro-2,2-dimethylbenzofuran-7-yl)-1-piperazinyl]-1-butyl]-2-imidazolidinone,1-[3-amino-2H-1-benzopyran-2-one-8-yl]piperazine, oxaflozane, RU-5031, CP-93129, naratriptan, avitriptan, rizatriptan, L-694247, IS-159, almotriptan, zolmitriptan, sumatriptan, eletriptan, VML-251, L-0076, ALX-0625, CP-122288, VS-395, L-747201, LY-334370 and salts thereof.

6. A composition according to claim 1 in which the 5-HT₁ agonist is a 5-HT_{1A} agonist.

7. A composition according to claim 6 in which the 5-HT₁ agonist is selected from buspirone, gepirone, ipsapirone, zalospirone, umespirone, alnespirone, enilospirone, WY-48723, SUN-8399, tandospirone and salts thereof.

8. A composition according to claim 7 in which the 5-HT₁ agonist is buspirone or a salt thereof.

9. A composition according to claim 1 in which the active ingredient is present in an amount of from 0.5 to 30% by weight of the composition.

10. A composition according to claim 1 in which the active ingredient is present in an amount of 0.25 to 50 mg.

11. A pharmaceutical composition for oral administration comprising a carrier, and buspirone as an active ingredient, characterized in that the composition is in the form of a solid fast-dispersing dosage form comprising a network of buspirone and a water-soluble or water-dispersible carrier which is inert towards buspirone, the network having been obtained by subliming solvent from a composition in the solid state, that composition comprising buspirone and a solution of the carrier in a solvent, wherein said solid fast-dispersing dosage form disintegrates within 1 to 10 seconds of being placed in the oral cavity.

12. A composition as defined in claim 1 for use in the treatment of anxiety.

13. Use of a composition as defined in claim 1 for the manufacture of a medicament for the treatment of depression.

14. Use of a composition as defined in claim 1 for the manufacture of a medicament for the treatment of Attention Deficit Disorder With or Without Hyperactivity.

15. Use of a composition as defined in claim 1 for the manufacture of a medicament for the treatment of panic disorders.

16. Use of a composition as defined in claim 1 for the manufacture of a medicament for use as a memory enhancer.

17. Use of a composition as defined in claim 1 for the manufacture of a medicament for the palliative treatment of anxiety neurosis.

18. Use of a composition as defined in claim 1 for the manufacture of a medicament for the treatment and/or prophylaxis of incontinence disorders.

19. Use of a composition as defined in claim 1 for the manufacture of a medicament for inducing immunosuppression and/or treating immune disorders.

20. Use of a composition as defined in claim 1 for the manufacture of a medicament for the treatment of sleep apnea and/or related respiratory disorders.

21. Use of a composition as defined in claim 1 for the manufacture of a medicament for the treatment for substance addiction.

22. Use of a composition as defined in claim 1 for the manufacture of a medicament for the treatment of alcohol abuse.

23. Use of a composition as defined in claim 1 for the manufacture of a medicament for the alleviation of extrapyramidal motor disorders.

24. Use of a fast-dispersing dosage form designed to release active ingredient within 1 to 60 seconds of being placed in the oral cavity to deliver a 5-HT₁ agonist.
